(19) United States
(12) Patent Application Publication
(10) Pub. No.: US 2017/0007618 A1
(43) Pub. Date: Jan. 12, 2017

(54) SEROTONIN 2C RECEPTOR ANTAGONISTS TO PREVENT AND TREAT STRESS-RELATED TRAUMA DISORDERS

(71) Applicant: Massachusetts Institute of Technology, Cambridge, MA (US)

(72) Inventors: Ki Ann Goosens, Cambridge, MA (US); Edward Stuart Boyden, Chestnut Hill, MA (US); Michael Vincent Baratta, Boulder, CO (US)

(73) Assignee: Massachusetts Institute of Technology, Cambridge, MA (US)

(21) Appl. No.: 15/185,628

(22) Filed: Jun. 17, 2016

Related U.S. Application Data
(60) Provisional application No. 62/181,073, filed on Jun. 17, 2015.

(51) Int. Cl. A61K 31/5513; A61K 9/00; C12N 15/113; A61K 31/165

(52) U.S. Cl. CPC A61K 31/5513; A61K 31/165; A61K 9/0053; C12N 15/1138; C12N 2320/31

(57) ABSTRACT

The invention relates to methods for preventing stress-associated disorders. These disorders may be treated with a serotonin 2c receptor (5-HT2CR) antagonist prior to, during, or following a stress-related event. Stress-associated disorders include, for instance, post-traumatic stress disorder (PTSD).

### RELATED APPLICATIONS

This Application claims priority under 35 U.S.C. §119(e) to U.S. Provisional Application Ser. No. 62/181,073, entitled “SEROTONIN 2C RECEPTOR ANTAGONISTS TO PREVENT AND TREAT STRESS-RELATED TRAUMA DISORDERS” filed on Jun. 17, 2015, which is herein incorporated by reference in its entirety.

### FEDERALLY SPONSORED RESEARCH

This invention was made with Government support under Grant Nos. OD002002 and MH084966 awarded by the National Institutes of Health and under Grant No. W81XWH-08-1-0401 awarded by the U.S Army Medical Research and Material Command and under Grant No. W911NF-10-1-0059 awarded the U.S. Army Research Office. The Government has certain rights in the invention.”

### BACKGROUND OF THE INVENTION

Stress exposure is a risk factor for the development of post-traumatic stress disorder (PTSD) in humans [1,2]. Humans with PTSD often have strong memories for the traumatic experiences that underlie their disorder [3], and also exhibit heightened fear conditioning in laboratory settings [4,5]. In preclinical studies, where exploration of the underlying neural mechanisms is feasible, the relationship between stress history and subsequent fear memory can be studied by exposing rodents to stressors and examining the impact on later Pavlovian fear conditioning. In these animal models, fear conditioning itself does not lead to PTSD; only stress-treated animals display the excessively strong fear memories that are also observed in humans with PTSD. The exaggerated fear response typically observed in stress-exposed animals [6] is often attributed to either strengthened encoding [7] or consolidation of the fear memory [8].

### SUMMARY OF INVENTION

Aspects of the invention include a method for preventing a stress-associated disorder that comprises administering to a subject at risk of having a stress-associated disorder a serotonin 2c receptor (5-HT2CR) antagonist prior to, during, or following a stress-related event in an effective amount to prevent the stress-associated disorder. In some embodiments, the stress-associated disorder is post-traumatic stress disorder (PTSD).

In some embodiments, the 5-HT2CR antagonist is agomelatine. In other embodiments, the 5-HT2CR antagonist is SB 242084, RS 102221 hydrochloride, SB 206553 {5-methyl-1-[(3-pyridylcarba-moyl)-1,2,3,5-tetrahydropyrrolo(2,3-f)indole]}, SB 206553 hydrochloride, SB 200646A [N-(1-methyl-5-indolyl)-N′-(3-pyridyl) urea hydrochloride], SB 200646 hydrochloride, clozapine, N-Desmethylclozapine, mesulergine hydrochloride, S 32212 hydrochloride, SB 221284, SDZ SER 082 fumarate, or analogs thereof.

In some embodiments, the 5-HT2CR antagonist is administered to the subject in an amount of 25 to 50 mg per day. In some embodiments, the 5-HT2CR antagonist is administered orally. In other embodiments, the 5-HT2CR antagonist is administered daily beginning one week prior to the stress-related event. In another embodiment, the 5-HT2CR antagonist is administered throughout the duration of the stress-related event. In some embodiments, the 5-HT2CR antagonist is administered for up to 24 weeks after the stress-related event.

Another aspect of the present disclosure includes a method for treating PTSD comprising administering to a subject having PTSD a 5-HT2CR antagonist in conjunction with a cognitive therapy for memory reconsolidation. In some embodiments, the 5-HT2CR antagonist is administered during the cognitive therapy for memory reconsolidation. In other embodiments, the 5-HT2CR antagonist is administered within 24 hours of the cognitive therapy for memory reconsolidation. In another embodiment, the 5-HT2CR antagonist is administered within 1 week of the cognitive therapy for memory reconsolidation.

In some embodiments, the 5-HT2CR antagonist is agomelatine. In other embodiments, the 5-HT2CR antagonist is administered to the subject in an amount of 25 to 50 mg per day. In another embodiment, the 5-HT2CR antagonist is administered orally. In other embodiments, the 5-HT2CR antagonist is not agomelatine. In some embodiments, the 5-HT2CR antagonist is a small molecule 5-HT2CR antagonist. In another embodiment, the 5-HT2CR antagonist is an inhibitory nucleic acid. In some embodiments, the 5-HT2CR antagonist is a 5HT2c receptor inverse agonist. In other embodiments, the 5-HT2CR inverse agonist is SB 228357 (N-[3-Fluoro-5-(3-pyrindyl)phenyl]-2,3-dihydro-5-methoxy-6-(trifluoromethyl)-1H-indole-1-carboxamide) or SB 243213 dihydrochloride (2,3-Dihydro-5-methyl-N-16-[(2-methyl-3-pyridinyl)oxy]-3-pyridinyl]-6-(trifluoromethyl)-1H-Indole-1-carboxamide dihydrochloride). In another embodiment, the 5-HT2CR antagonist is a clozapine metabolite. In some embodiments, the clozapine metabolite is clozapine N-oxide.

Another aspect of the present disclosure encompasses a method for treating or preventing a stress-associated disorder comprising administering to a subject having a stress-associated disorder a 5-HT2CR antagonist in an effective amount to treat the stress-associated disorder, wherein the 5-HT2CR antagonist is not agomelatine.

In some embodiments, the 5-HT2CR antagonist is a small molecule 5-HT2CR antagonist. In other embodiments, the 5-HT2CR antagonist is an inhibitory nucleic acid. In another embodiment, the 5-HT2CR antagonist is a 5HT2CR inverse agonist. In some embodiments, the 5-HT2CR inverse agonist is SB 228357 or SB 243213 dihydrochloride. In other embodiments, the 5-HT2CR antagonist is a clozapine metabolite. In another embodiment, the clozapine metabolite is clozapine N-oxide.

Aspects of the present disclosure include a method for treating or preventing a stress-associated disorder comprising identifying a subject who has enhanced fear associated with multiple stresses or traumatic memory strength and administering to the subject a 5-HT2CR antagonist in an effective amount to treat or prevent the stress-associated disorder. In some embodiments, the subject who has enhanced fear associated with multiple stresses or traumatic memory strength is a subject selected from the group consisting of a soldier deployed to an active combat zone, a subject living in an active war zone, and a first responder.

In some embodiments, the 5-HT2CR antagonist is agomelatine. In other embodiments, the 5-HT2CR antagonist is SB 242084, RS 102221 hydrochloride, SB 206553 {5-methyl-1-[(3-pyridylcarba-moyl)-1,2,3,5-tetrahydropyrrolo(2,3-f)indole]}, SB 206553 hydrochloride, SB 200646A [N-(1-methyl-5-indolyl)-N′-(3-pyridyl) urea hydrochloride], SB 200646 hydrochloride, clozapine, N-Desmethylclozapine, mesulergine hydrochloride, S 32212 hydrochloride, SB 221284, SDZ SER 082 fumarate, or analogs thereof.

In some embodiments, the 5-HT2CR antagonist is administered to the subject in an amount of 25 to 50 mg per day. In other embodiments, the 5-HT2CR antagonist is administered orally.

In some embodiments, the 5-HT2CR antagonist is not agomelatine. In other embodiments, the 5-HT2CR antagonist is a small molecule 5-HT2CR antagonist. In another embodiment, the 5-HT2CR antagonist is an inhibitory nucleic acid. In other embodiments, the 5-HT2CR antagonist is a 5HT2CR inverse agonist. In another embodiment, the 5-HT2CR inverse agonist is SB 228357 or SB 243213 dihydrochloride. In some embodiments, the 5-HT2CR antagonist is a clozapine metabolite. In other embodiments, the clozapine metabolite is clozapine N-oxide.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1B show that stress recruits serotonergic fear memory consolidation. FIG. 1A shows that prior stress did not impact short-term (2 h) fear memory (left), but increased long-term (24 h) fear memory (right) to the tone. FIG. 1B shows that post-conditioning infusion of the serotonin 2C receptor antagonist SB242084 into the lateral/basolateral amygdala [24] blocked the stress-induced enhancement of fear consolidation. Data are means±s.e.m. Fisher's PLSD comparisons during auditory fear test: *P<0.05 and n.s.=not significant for Stress versus No Stress.

FIG. 2 shows that stress does not affect conditioning-related increases in amygdalar serotonin. Fear conditioning produced a significant elevation in serotonin (5-HT) in the BLA, but this was not altered by previous stress exposure. Data are means±s.e.m. Fisher's PLSD comparisons to the Home Cage group: * P<0.05.

FIGS. 3A-3C show that stress enhances surface expression of 5-HT2C receptors in BLA. Stress enhanced membrane expression of the 5-HT2C receptor in the BLA (FIG. 3A) without affecting the total levels of 5-HT2C receptors (FIG. 3B), suggesting a change in trafficking of the receptor. FIG. 3C shows that stress also produced a concurrent increase in the mRNA editing enzyme ADAR1 in the BLA. Images on the right depict all bands in representative samples. Data are means±s.e.m. Fisher's PLSD comparisons: * P<0.05

FIGS. 4A-4B show that prior stress enhances fear to unambiguous cues. FIG. 4A shows that prior stress enhances tone-elicited freezing in a conditioning paradigm with a tone-footshock contingency of 100%, but no facilitation was observed when repeated stress followed conditioning (n=8-10/group) (FIG. 4B). Data are means±s.e.m. Fisher's PLSD group comparisons during tone fear test: * P<0.05 and n.s.=not significant for Stress versus No Stress.

FIGS. 5A-5D show that prior stress does not alter freezing or pain sensitivity during conditioning or general motor activity prior to fear retrieval. During the acquisition phase, the level of freezing behavior (FIG. 5A) and the motor response (FIG. 5B) evoked by the conditioning footshocks did not differ between Stress and No Stress groups (n=27-29/group). During the pre-tone period of the auditory fear test, the total distance (FIG. 5C) and velocity (FIG. 5D) of motor activity did not differ between Stress and No Stress groups. Data are means±s.e.m.

FIG. 6 shows that repeated stress must precede conditioning in order to impact fear expression. Stress given after fear conditioning did not alter retrieval of the long-term auditory fear memory. Data are means±s.e.m. Fisher's PLSD comparisons during auditory fear test: n.s.=not significant for Stress versus No Stress.

FIG. 7 shows that acute stress does not alter long-term fear memory. A single session of immobilization stress prior to fear conditioning did not augment long-term fear memory (n=9-10/group). Data are means±s.e.m. Fisher's PLSD group comparisons during tone fear test: n.s.=not significant for Stress versus No Stress.

### DETAILED DESCRIPTION

Repeated exposure to stress produces a vulnerability to heightened fear learning. It is demonstrated herein that this vulnerability emerges from a serotonergic fear memory consolidation process that is not present in unstressed subjects. This serotonergic consolidation process requires serotonergic activity in the dorsal raphe nucleus (DRN) during aversive reinforcement and 5-HT2CR signaling in the basolateral amygdala (BLA), a major target structure of the DRN [20-24]. Interestingly, it has also been discovered herein that serotonin activation by either signaled or unsignaled footshocks is sufficient to enhance associative fear memory in a stressed subject, an effect not predicted by classic theoretical models of associative learning. We show that stress enhances cell surface expression of 5-HT2CRs in the amygdala without affecting total serotonin levels during fear conditioning. Thus, aversive reinforcement is processed differently in the brain of a stress-exposed subject than a subject who has not been exposed to stress. This profoundly impacts the memory of aversive experiences. These findings reveal fundamental mechanisms underlying the operation of a critical neural system in affective processing, and provide new principles both the prevention of stress-related psychiatric disorders.

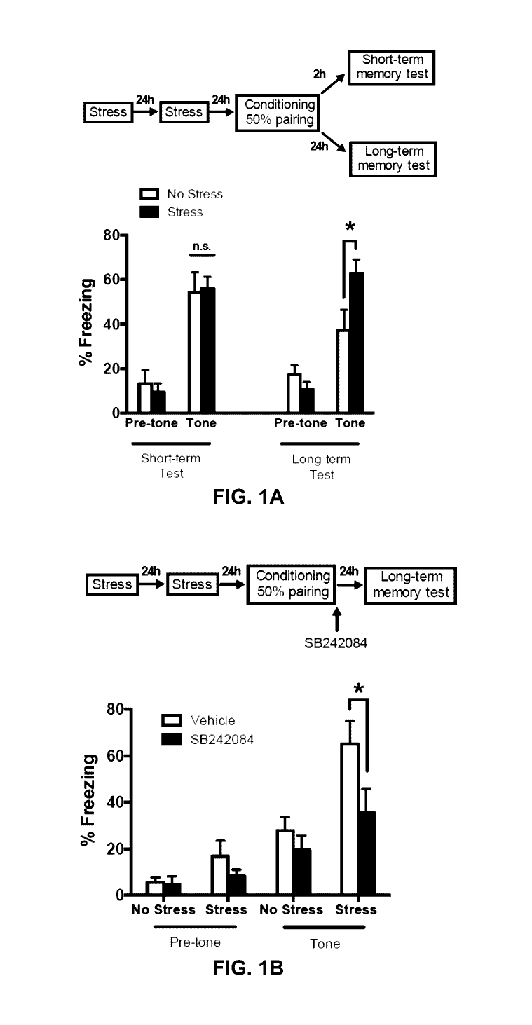

A surprising finding of the invention involves the discovery that serotonergic fear memory consolidation was only observed in a subject with a history of repeated stress exposure. This was demonstrated, as shown in the Examples section below, by the selective reduction of fear in stressed, but not unstressed, mice by post-conditioning intra-BLA infusion of a 5-HT2CR antagonist (FIG. 1B). It has been demonstrated according to the invention that stress increases the expression of 5-HT2CR membrane receptors in the BLA, and this shows a mechanism by which 5-HT2CR-dependent fear memory consolidation is engaged following stress exposure.

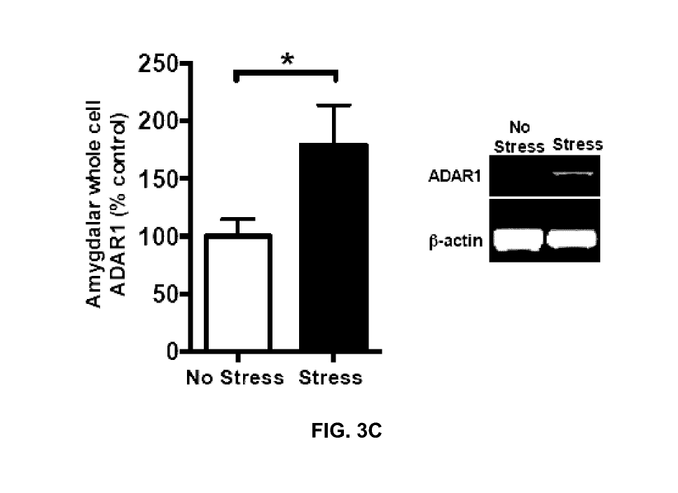

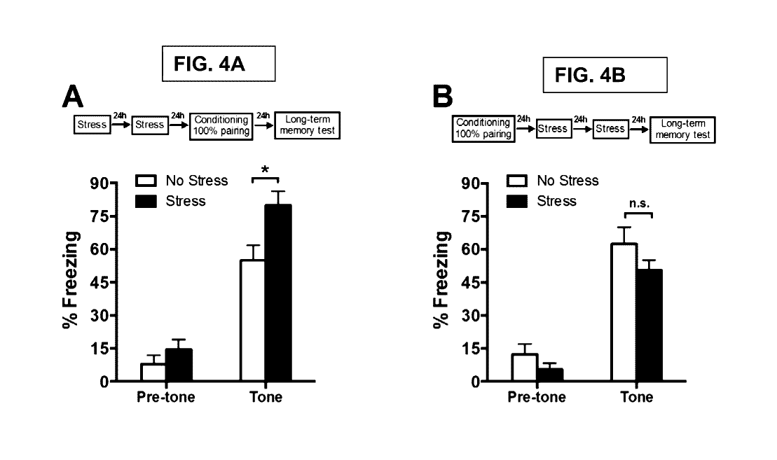

While aversive reinforcement triggers activity in serotonergic neurons [12] (FIG. 4), it is clear that synaptic serotonin can remain elevated in projection regions such as the BLA for at least an hour following conditioning [14]. Thus, while serotonin may bind to its receptors during fear learning, it is also capable of binding during a brief (˜hours) post-training consolidation window. The data described herein that stress enhances long-term, but not short-term, fear memory (FIGS. 1A, 5A) via post-conditioning activity at 5-HT2CRs in the BLA (FIG. 1B) are consistent with this.

Thus, during fear learning, serotonergic neurons make a critical contribution to the fear-enhancing effect of stress, elicited by the presentation of aversive stimuli during fear conditioning. Furthermore, this effect is mediated by postsynaptic actions at 5-HT2CRs in the BLA, which enhance fear memory consolidation. These results show that while the triggers leading to serotonin release (i.e., presentation of aversive stimuli) are temporally delimited, the effects of serotonin on downstream targets like the BLA are persistent. This mechanism may explain why polymorphisms in human serotonergic genes are often associated with enhanced aversive processing, especially following a history of traumatic life events [10,51,52].

Thus, in some aspects, the invention is a method for treating or preventing a stress-associated disorder, by administering to a subject having or at risk of having a stress-associated disorder a 5-HT2CR antagonist in an effective amount to treat the stress-associated disorder.

The 5-HT2CR antagonist is useful for preventing the development of the stress-associated disorder and for treating the stress-associated disorder. In some instances, the antagonist is administered to the subject either prior to or during the stress exposure, or immediately following the stress exposure.

5-HT2CR is a subtype of 5-HT ((5-hydroxytryptamine) receptor for the endogenous neurotransmitter serotonin. The receptor is a G protein-coupled receptor (GPCR). A 5-HT2CR antagonist, as used herein, refers to a compound that prevents, inhibits or reduces to any extent activation or expression of the 5-HT2CR. The compound that prevents or inhibits activation of the 5-HT2CR may act directly or indirectly on the 5-HT2CR. For example the compound may bind or interact directly with the 5-HT2CR in some embodiments. In other embodiments the compound may act indirectly by blocking access of the endogenous neuronal serotonin to the 5-HT2CR or by limiting the expression of active 5-HT2CR in neuronal cells. For instance the compound may be able to block access of the endogenous neuronal serotonin to the 5-HT2CR by blocking the 5-HT2CR binding site on serotonin or the serotonin binding site on 5-HT2CR.

5-HT2CR antagonists include small molecule, protein and nucleic acid 5-HT2CR antagonists. 5-HT2CR antagonists are well known in the art and include, but are not limited to agomelatine (N-[2-(7-methoxynaphthalen-1-yl)ethyl]acetamide, sold under trade names including: Melitor, Thymanax, and Valdoxan), SB 242084 (6-chloro-5-methyl-N-{6-[(2-methylpyridin-3 -yl)oxy]pyridin-3-yl}indoline-1-carboxamide), RS 102221 hydrochloride (8-[5-(2,4-Dimethoxy-5-(4-trifluoromethylphenylsulphonamido)phenyl-5-oxopentyl]-1,3,8-triazaspiro[4.5]decane-2,4-dione hydrochloride), SB 206553 {5-methyl-1-[(3-pyridylcarba-moyl)-1,2,3,5-tetrahydropyrrolo(2,3-f)indole]}, SB 206553 hydrochloride (3,5-Dihydro-5-methyl-N-3-pyridinylbenzo[1,2-b:4,5-b′]dipyrrole-1(2H)-carboxamide hydrochloride), SB 200646A [N-(1-methyl- 5-indolyl)-N′-(3-pyridyl) urea hydrochloride], SB 200646 hydrochloride (N-(1-Methyl-1H-indo1-5-yl)-N′-3-pyridinylurea), clozapine, N-Desmethylclozapine, mesulergine hydrochloride, S 32212 hydrochloride (1,2-Dihydro-N-[4-methoxy-3-(4-methyl-1-piperazinyl)phenyl]-3H-benz[e]indole-3-carboxamide hydrochloride), SB 221284 (2,3-Dihydro-5-(methylthio)-N-3-pyridinyl-6-(trifluoromethyl)-1H-indole-1-carboxamide), SDZ SER 082 fumarate ((+)-cis-4,5,7a,8,9,10,11,11a-Octahydro-7H-10-methylindolo[1,7-bc][2,6] -naphthyridine fumarate), SB 228357 (N-[3-Fluoro-5-(3-pyrindyl)phenyl]-2,3-dihydro-5-methoxy-6-(trifluoromethyl)-1H-indole-1-carboxamide), SB 243213 dihydrochloride (2,3-Dihydro-5-methyl-N-[6-[(2-methyl-3-pyridinyl)oxy]-3-pyridinyl]-6-(trifluoromethyl)-1H-Indole-1-carboxamide dihydrochloride) or analogs thereof.

SB 242084 is one of the most potent and selective 5-HT2C receptor antagonist available. RS 102221 hydrochloride is another highly potent antagonist. Historically, the overall sequence identity between 5-HT2C other 5-HT2 receptor subtypes (5-HT2A, 5-HT2B) has made the development of agonists/antagonists selective to the 2C receptor difficult. That is, most 2A and 2B antagonists also have some affinity to 2C. SB 242084 has a 158- and 100-fold selectivity over 5-HT2A and 5-HT2B receptors respectively (RS 102221 has a 100-fold selectivity over 2A and 2B).

SB 206553 {5-methyl-1-[(3-pyridylcarba-moyl)-1,2,3,5-tetrahydropyrrolo(2,3-f)indole]} (pKᵢ 7.9) and SB 200646A [N-(1-methyl-5-indolyl)-N′-(3-pyridyl) urea hydrochloride] (pKᵢ 6.9) are also highly selective. As a comparison, SB 242084 has a pKᵢ 9.0 and RS 102221 has a pKᵢ 8.7 for the cloned human 5-HT2C receptor.

Thus, in some embodiments the antagonists are selective antagonists. A selective 5-HT2CR antagonist is one which is selective for the 5-HT2CR over the highly homologous 5-HT2AR and 5-HT2BR. Ligands of the 5-HT2AR and 5-HT2BRs can produce adverse CNS and cardiovascular events that are not associated with selective antagonism of the 5-HT2CR.

In some embodiments the 5-HT2CR antagonist is a 5-HT2CR inverse agonist or an inhibitory nucleic acid. The 5-HT2CR antagonist that is an inhibitory nucleic acid may be, for instance, an siRNA or an antisense molecule that inhibits expression of a 5-HT2CR or a gene editing toolkit. The nucleic acid sequence of 5-HT2CR is well known in the art. See for instance, Gene ID:3358 in NCBI database as well as in Xie E1, et al. Genomics. The human serotonin 5-HT2C receptor: complete cDNA, genomic structure, and alternatively spliced variant. 1996 Aug. 1; 35(3):551-61. The inhibitory nucleic acids may be designed using routine methods in the art.

The 5-HT2CR antagonists do not include compounds that are serotonin reuptake inhibitors that function through receptors other than the 5-HT2CR. In some embodiments the 5-HT2CR antagonists do not include serotonin reuptake inhibitors at all.

In some embodiments, the 5-HT2CR antagonist has more than 5-fold selectivity, more than 10-fold selectivity, more than 20-fold selectivity, more than 30-fold selectivity, more than 40-fold selectivity, more than 50-fold selectivity, more than 60-fold selectivity, more than 70-fold selectivity, more than 20-fold selectivity, more than 80-fold selectivity, more than 90-fold selectivity, more than 100-fold selectivity, or more than 150-fold selectivity over 5-HT2a and/or 5-HT2b receptors. In certain embodiments, the 5-HT2CR antagonist has like selectivity over other 5-HT, dopamine and adrenergic receptors.

A 5-HT2CR inhibitory nucleic acid typically causes specific gene knockdown, while avoiding off-target effects. Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. In one embodiment, vector-based RNAi modalities (e.g., shRNA expression constructs) are used to reduce expression of a gene (e.g., a target nucleic acid such as a 5-HT2CR nucleic acid) in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a specific promoter operably linked to a gene encoding the small interfering nucleic acid. In some cases, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting and siRNAs with ribo-difluorotoluyl nucleotides maintain gene silencing activity. siRNAs with amide-linked oligoribonucleosides have been generated that are more resistant to S1 nuclease degradation than unmodified siRNAs. In addition, modification of siRNAs at the 2′-sugar position and phosphodiester linkage confers improved serum stability without loss of efficacy. Other molecules that can be used to inhibit expression of a gene include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. The diverse array of suppression strategies that can be employed includes the use of DNA and/or RNA aptamers that can be selected to target a protein of interest (e.g, 5-HT2CR).

Other inhibitor molecules that can be used include sense and antisense nucleic acids (single or double stranded). Antisense nucleic acids include modified or unmodified RNA, DNA, or mixed polymer nucleic acids, and primarily function by specifically binding to matching sequences resulting in modulation of peptide synthesis. Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm.

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

In some embodiments the inhibitory nucleic acid of the invention is 100% identical to the nucleic acid target. In other embodiments it is at least 99%, 95%, 90%, 85%, 80%, 75%, 70%, or 50% identical to the nucleic acid target. The term “percent identical” refers to sequence identity between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ-FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

An inhibitory nucleic acid useful in the invention will generally be designed to have partial or complete complementarity with one or more target genes (i.e., complementarity with one or more transcripts of 5-HT2CR gene). The target gene may be a gene derived from the cell, an endogenous gene, a transgene, or a gene of a pathogen which is present in the cell after infection thereof. Depending on the particular target gene, the nature of the inhibitory nucleic acid and the level of expression of inhibitory nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein. “Inhibition of gene expression” refers to the absence or observable decrease in the level of protein and/or mRNA product from a target gene. “Specificity” refers to the ability to inhibit the target gene without manifest effects on other genes of the cell.

Aspects of the invention relate to the effects of stress and, in particular, chronic stress. As used herein, “stress” refers to a physical, chemical or emotional factor or combination of factors that causes bodily or mental tension and that may be a factor in disease causation. It should be appreciated that any form of stress can be compatible with aspects of the invention. Exposure to stress can be chronic or acute. As used here, “chronic stress” refers to a state of prolonged tension from internal or external stressors, which may cause various physical manifestations. The effects of chronic and acute stress can be different. Several non-limiting examples of situations where a subject could be exposed to chronic stress include military service such as a combat mission, and natural disasters, such as participation in a search-and-rescue operation or rebuilding following a natural disaster. These are encompassed within the definition of stress-associated disorders, as used herein.

Subjects who are exposed to stress can also develop stress-sensitive disorders. As used herein, a “stress-sensitive disorder” refers to any condition, disease or disorder that results, at least in part, from exposure to stress or is exacerbated, at least in part, from exposure to stress. Non-limiting examples of stress-sensitive disorders include Post-traumatic Stress Disorder (PTSD), Bipolar Disorder, Acute Stress Disorder, anxiety disorders such as Generalized Anxiety Disorder, Obsessive-Compulsive Disorder, social anxiety disorders, Panic Disorders, schizophrenia, phobias, obsessive compulsive disorders, and Trichotillomania. It should be appreciated that any stress-sensitive disorder can be compatible with aspects of the invention.

Post-Traumatic Stress Disorder (PTSD) is mental health condition caused by exposure to psychological damage by experience beyond a usual corrective ability such as traumas of wars, natural disasters, domestic violence or sexual abuse, etc. It is believed that in addition to psychological manifestations, shrinkage of the hippocampus and dysfunction of prefrontal cortex often occurs. The principal characteristic symptoms involve re-experiencing a traumatic (i.e., psychologically distressing) event, the avoidance of stimuli associated with that event, the numbing of general responsiveness, and increased arousal. The “events” concerned are outside the range of common experiences such as simple bereavement, chronic illness and marital conflict.

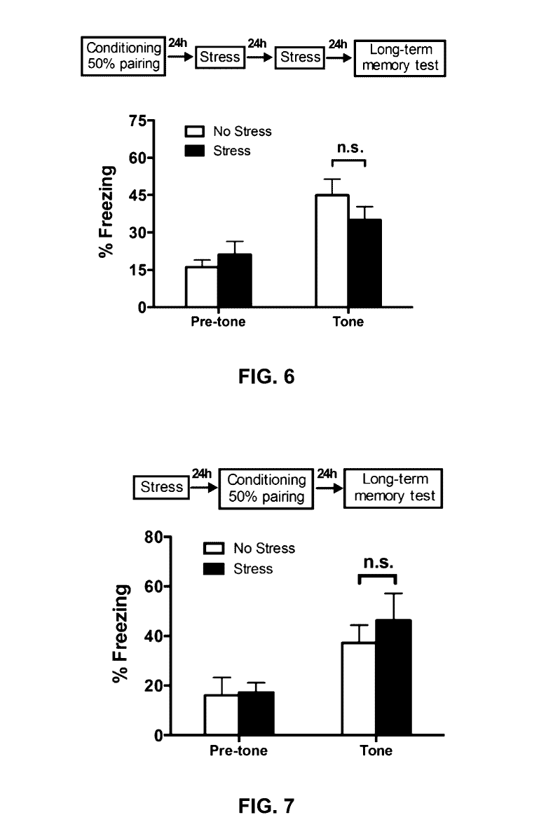

The data presented herein on treatment and prevention of PTSD involves the use of a rodent model of PTSD that captures critical features of the disorder. First, the strong fear memory produced by the conditioning experience in stressed animals mirrors the strong memories for traumatic events often observed in humans with PTSD [53]. While PTSD involves additional symptoms, the intrusive and powerful nature of the traumatic memory may contribute to some other symptoms, such as hypervigilance or sleep disturbance [3,54]. Also, the dose-response relationship between stress exposure and enhancement of fear observed in our model (FIGS. 1, 7) parallels the relationship between stress exposure and vulnerability to PTSD in humans [55]. The demonstration that pharmacological and optogenetic inhibition of a serotonergic subcircuit selectively reduces fear in stressed animals with “pathological” (exaggerated) fear levels, without affecting fear responding in unstressed animals, overcomes a critical barrier to the successful treatment of stress-induced anxiety disorders such as PTSD. The benchmark for the successful treatment of PTSD should not be the elimination of fear, but simply its reduction to normal, adaptive levels. The findings of the invention indicate that administration of a 5-HT2CR antagonist should be useful in the prevention or treatment of PTSD by reducing the consolidation or reconsolidation of fear memory.

Phobias include specific phobias and social phobias. Specific phobia is an anxiety disorder of which the essential feature is a persistent fear of a circumscribed stimulus, which may be an object or situation, other than fear of having a panic attack or of humiliation or embarrassment in social situations (which falls under social phobia). Examples include phobias of flying, heights, animals, injections, and blood. Simple phobias may be referred to as “specific” phobias and, in the population at large. Exposure to the phobic stimulus will almost invariably lead to an immediate anxiety response. Social phobia is characterized by the persistent fear of social or performance situations in which embarrassment may occur.

Aspects of the invention relate to methods by which the effects of recurring stress can be weakened to reduce the potentiating effects of stress on stress-sensitive mental illnesses. Methods associated with the invention comprise administration of a therapeutically effective amount of a 5-HT2CR antagonist to a subject.

The 5-HT2CR antagonist can be administered to a subject before, during and/or after exposure to chronic stress. For example, the 5-HT2CR antagonist can be administered to a subject in anticipation of exposure to chronic stress, such as prior to participation in a military operation. As such, the 5-HT2CR antagonist can protect against the consequences of exposure to chronic stress. The 5-HT2CR antagonist can also be administered to a subject during exposure to chronic stress to protect against the consequences of exposure to chronic stress and treat symptoms associated with the effects of the stress. The 5-HT2CR antagonist can also be administered after, and especially immediately after (i.e. within 24 hours, within 20 hours, within 15 hours, within 12 hours, within 10 hours, within 5 hours, within 4 hours, within 3 hours, within 2 hours, within 1 hour following the stress-related event) to protect against the consequences of exposure to chronic stress and treat symptoms associated with the effects of chronic stress.

Administering a 5-HT2CR antagonist to a subject who will be exposed to chronic stress may reduce the incidence of trauma-induced disorders such as post-traumatic stress disorder (PTSD). Moreover, in the past, most stress-sensitive illnesses have been treated with the same compounds that are used to treat other mental illnesses, such as selective serotonin reuptake inhibitors (SSRIs). However, these drugs do not offer any clinical benefit to a significant number of patients diagnosed with these disorders. Having drugs with a novel mechanism of action, targeting the 5-HT2CR signaling pathway, may be beneficial for patients who are resistant to traditional avenues of treatment.

The methods of the invention are useful for treating a subject in need thereof. A subject in need thereof can be a subject who will be exposed to chronic stress, is currently exposed to chronic stress or has been exposed to chronic stress. For example, a subject in need thereof may be a subject involved, or who will be involved, in a military operation or combat mission. A subject in need thereof can be a subject having or at risk of a stress-associated disorder. For example, a subject can be a patient who is diagnosed with a stress-sensitive disorder, or a subject with a strong familial history of such disorders.

In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms associated with the disorder or the severity of the disease or preventing any further progression of the disease. If the subject in need of treatment is one who is at risk of having a condition, then treating the subject refers to reducing the risk of the subject having the condition or preventing the subject from developing the condition.

The methods of the invention are also useful for preventing a stress-associated disorder by administering the 5-HT2CR antagonist to a subject at risk of developing the disorder. The term prevent refers to a prophylactic treatment.

A subject shall mean a human or vertebrate animal or mammal including but not limited to a dog, cat, horse, cow, pig, sheep, goat, turkey, chicken, and primate, e.g., monkey.

Therapeutic compounds associated with the invention may be directly administered to the subject or may be administered in conjunction with a delivery device or vehicle. Delivery vehicles or delivery devices for delivering therapeutic compounds to surfaces have been described. The therapeutic compounds of the invention may be administered alone (e.g., in saline or buffer) or using any delivery vehicles known in the art.

The term effective amount of a therapeutic compound of the invention refers to the amount necessary or sufficient to realize a desired biologic effect. For example, an effective amount of a therapeutic compound associated with the invention may be that amount sufficient to ameliorate one or more symptoms of a stress-associated disorder in a subject who has been exposed to chronic stress. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the invention without necessitating undue experimentation.

Subject doses of the compounds described herein for delivery typically range from about 0.1 μg to 10 mg per administration, which depending on the application could be given daily, weekly, or monthly and any other amount of time there between. The doses for these purposes may range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

In some embodiments a compound of the invention is administered at a dosage of between about 1 and 10 mg/kg of body weight of the mammal. In other embodiments a compound of the invention is administered at a dosage of between about 0.001 and 1 mg/kg of body weight of the mammal. In yet other embodiments a compound of the invention is administered at a dosage of between about 10-100 ng/kg, 100-500 ng/kg, 500 ng/kg-1 mg/kg, or 1-5 mg/kg of body weight of the mammal, or any individual dosage therein.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compound associated with the invention can be administered to a subject by any mode that delivers the therapeutic agent or compound to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, rectal and intracerebroventricular.

For oral administration, the therapeutic compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline (Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the invention. The therapeutic agent is delivered to the lungs of a mammal while inhaling and traverses across the lung epithelial lining to the blood stream. Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified therapeutic agent may also be prepared in different formulations depending on the type of chemical modification or the type of device employed.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Intra-nasal delivery of a pharmaceutical composition of the present invention is also contemplated. Intra-nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a therapeutic compound of the invention optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agents may be delivered to the brain using a formulation capable of delivering a therapeutic agent across the blood brain barrier. One obstacle to delivering therapeutics to the brain is the physiology and structure of the brain. The blood-brain barrier is made up of specialized capillaries lined with a single layer of endothelial cells. The region between cells is sealed with a tight junction, so the only access to the brain from the blood is through the endothelial cells. The barrier allows only certain substances, such as lipophilic molecules through and keeps other harmful compounds and pathogens out. Thus, lipophilic carriers are useful for delivering non-lipophilic compounds to the brain. For instance, DHA, a fatty acid naturally occurring in the human brain has been found to be useful for delivering drugs covalently attached thereto to the brain (Such as those described in U.S. Pat. No. 6,407,137). U.S. Pat. No. 5,525,727 describes a dihydropyridine pyridinium salt carrier redox system for the specific and sustained delivery of drug species to the brain. U.S. Pat. No. 5,618,803 describes targeted drug delivery with phosphonate derivatives. U.S. Pat. No. 7119074 describes amphiphilic prodrugs of a therapeutic compound conjugated to an PEG-oligomer/polymer for delivering the compound across the blood brain barrier. Others are known to those of skill in the art.

The therapeutic agents of the invention may be delivered with other therapeutics for treating stress-associated disorders.

The phraseology and terminology used herein is for the purpose of description and should not be regarded as limiting. The use of “including,” “comprising,” “having,” “containing,” “involving,” and variations thereof, is meant to encompass the items listed thereafter and additional items. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

### Materials and Methods

Subjects. Adult male C57BL/6 mice (Taconic, Germantown, N.Y.) or transgenic mice expressing Cre recombinase under the transcriptional control of the serotonin transporter promoter (SERT-Cre; provided by a collaborator) [25] were used in all experiments. SERT-Cre mice were backcrossed to C57BL/6 for at least seven generations prior to experimental use. Food and water were provided ad libitum. Mice (6-8 weeks old at the time of experimentation) were allowed to acclimate to colony conditions (68-72° F.; 12-h light-dark cycle, 7 AM lights on) for 7-10 days prior to the start of experimental procedures. All mice were group-housed (4-5/cage). For experiments in which surgery was conducted, mice were singly housed post-surgery. All procedures were approved by the Committee on Animal Care at the Massachusetts Institute of Technology and the Animal Care and Use Review Office at the U.S. Army Medical Research and Material Command.

Virus. To construct adeno-associated viral (AAV) vectors, a flip-excision (FLEX) switch carrying two pairs of antiparallel loxP-type recombination sites (loxP and lox2722) was synthesized and transgenes encoding archaerhodopsin-3 fused with green fluorescent protein (Arch-GFP) or GFP alone (control) were inserted between the loxP and lox2722 sites in the reverse orientation. AAV vectors were serotyped with AAV 2/8 capsids and packaged by the Vector Core at The University of North Carolina at Chapel Hill. The final viral concentration was approximately 1.0-2.0×1011 infectious particles/mL.

Drugs. The selective 5-HT2CR antagonist 6-chloro-2,3-dihydro-5-methyl-N-[6-[(2-methyl-3-pyridinyl) oxy]-3-pyridinyl]-1H-indole-1-carboxyamide dihydrochloride (SB242084, Tocris Bioscience, Minneapolis, Minn.) was dissolved in 0.9% sterile saline.

Viral delivery and optical fiber implantation. Under isoflurane anesthesia (Webster Veterinary, Devens, Mass.), Cre-dependent AAV vectors carrying archaerhodopsin-3 fused with green fluorescent protein (FLEX-Arch-GFP) or control FLEX-GFP constructs were injected into the dorsal raphe nucleus (DRN; 4.4 mm posterior to bregma, 1.5 mm relative to the midline, and 2.5 mm ventral to the cortical surface, at a 20° angle to avoid puncturing the sinus) in transgenic mice expressing Cre recombinase under the transcriptional control of the serotonin transporter promoter (SERT-Cre). Virus was delivered to the DRN using a 10-μl syringe and a thin 33-gauge metal needle with a beveled tip (Hamilton Company, Reno, Nev.). The injection volume (1.0 μl) and flow rate (0.1 μl/min) were controlled with a microinjection pump (World Precision Instruments, Sarasota, Fla.). Following injection, the needle was left in place for an additional 10 min to allow diffusion of the virus.

For behavioral experiments, a multimode optical fiber (200 μm diameter core, NA 0.48; Thorlabs, Newton, N.J.) coupled to a ceramic ferrule (225 μm diameter core; Kientec Systems Inc., Stuart, Fla.) was implanted over the same stereotactic coordinates as mentioned above. The optical fiber implant was secured to the skull with stainless steel screws and dental cement. SERT-Cre mice were allowed to recover for at least 3 weeks before behavioral and electrophysiological experimentation.

Cannula implantation and microinfusion. In C57BL/6 mice, stainless steel acute guide cannulae (26 gauge; Plastics One, Roanoke, Va.) were targeted unilaterally to the DRN (4.4 mm posterior to bregma, 1.5 mm relative to the midline, and 2.5 mm ventral to the cortical surface) at a 20° angle or bilaterally to the basolateral amygdala (BLA; 1.4 mm posterior to bregma, ±3.1 mm relative to the midline, and 3.8 mm ventral to the cortical surface). The cannulae were secured with stainless steel screws and dental cement. SB242084, a selective serotonin 2c receptor (5-HT2CR) antagonist, was delivered to the BLA immediately following fear conditioning. Drug administration was controlled by a programmable microinjection pump (Harvard Apparatus, Holliston, Mass.) that delivered drugs to the injection site over a one-minute period (SB242084: 0.4 μg/0.4 μl). Microinfusion volumes for these structures were similar to those previously reported [63-64]. The injector was left in place for an additional minute to allow diffusion from the needle tip before the injector was removed.

Fear conditioning apparatus. Conditioning occurred in clear plastic chambers (10 L×8 W×7 H inch) that were placed in a sound-attenuating cabinet. The cabinet had a tone generator and a 15 W clear light bulb mounted to the ceiling. The conditioning chambers rested on a removable floor of stainless-steel rods (ENV-3013WR; Med Associates, St. Albans, Vt.). Each rod was wired to a shock generator and scrambler (ENV-414S; Med Associates) for the delivery of footshock. The mounted tone generator delivered an 85 db, 2.2 kHz tone. Presentation of stimuli was delivered via a TTL pulse generator (National Instruments, Austin, Tex.) and controlled with Python 2.6 software.

Fear conditioning. Prior to conditioning, each mouse was taken from its colony room and transported to a holding room for 1 h. Fear conditioning and testing took place in a room separate from that where immobilization occurred. The fear conditioning protocol consisted of 4 tone conditional stimulus presentations (CS; each 30 sec in duration) and 4 footshock unconditional stimulus presentations (US; 0.5 mA, each 2 sec in duration). The first CS presentation always occurred 2 min after placement of the subject in the conditioning chamber, and a 2 min interval separated all CSs and concluded the session. Importantly, the session duration, the number of CS presentations, and the number of US (Unconditional stimulus) presentations was the same for all subjects. To achieve 50% CS-US pairing, two of the US s were paired with CSs (the 2-sec footshock coincided with the last 2 seconds of the 30-sec tone), while the remaining two US presentations occurred during the inter-CS intervals either 42, 52, or 72 seconds prior to the next CS presentation. To achieve 0% CS-US pairing, all four US presentations were presented during the inter-CS intervals, either 42, 52, or 72 seconds prior to the next CS presentation.

To measure auditory fear memory strength, mice were returned the following day to an altered context. In the novel test environment the original conditioning chamber was altered by removal of the shock grid and placement of a Plexiglas plate between two diagonally opposite corners, forming a triangular chamber. The brightly lit conditioning chamber was replaced with a 25 W red light bulb. Further, the house light for the room was turned off. During the initial 3 min (pre-tone) the subject's freezing to the novel environment was scored. This was followed by presentation of the conditional tone for 3 min. Freezing was defined as the absence of all movement except that required for respiration [65]. For some experiments, behavior during the tone test was recorded by a digital video camera mounted directly above the chamber and freezing levels were scored by a male observer blind to the experimental groups using a time-sampling procedure every 10 seconds throughout the memory test. In some experiments, an infrared camera recorded behavior during conditioning and the tone test, and activity levels were determined with software using a proprietary formula that calculates a value (30 Hz) for the average change of grayscale pixel values in the video (VideoFreeze, Med Associates). In this case, the time spent freezing was calculated by the software after the experimenter determined a “threshold” value for freezing. Percent freezing was computed for each tone presentation and during 1 min bins before the presentation of the first tone; this yields an index of fear memory strength amenable to parametric statistics [65]. For assessing shock reactivity, the average raw value of the pixel change was used as a measure of motor activity (arbitrary units) during each 2 sec shock.

Photoinhibition. For Arch-mediated photoinhibition, a 532 nm green laser diode (Shanghai Laser & Optics Century Co., Shanghai, China) was coupled to a 200-μm multimode silica-core optical fiber through an FC/PC adapter. A fiber-optic rotary joint (Doric Lenses, Quebec, Canada) was used to release torsion in the connector fiber caused by the animal's rotation. Photostimuli consisted of green light pulses of 30-sec duration and power levels that yielded a fiber tip irradiance approximately 225 mW/mm² as determined by an optical power meter (Newport, Irvine, Calif.).

Immobilization stress. Mice were transferred to an experimental room and placed for one hour in ventilated plastic Decapicone bags (Braintree Scientific, Braintree, Mass.) for two consecutive days.

ELISA. Thirty minutes after fear conditioning, mice were overdosed with isoflurane and the brain was rapidly dissected and placed into chilled 0.1 M phosphate-buffered saline (pH 7.4) for one minute. After placement in a chilled matrix, 1 mm thick coronal sections were taken. Bilateral punches (2 mm diameter) containing the BLA were removed from each mouse and placed in a low-binding Eppendorf tube, flash frozen, and stored at −80 ° C.

Tissue was thawed on ice and homogenized using a motorized pestle (VWR, Radnor, Pa.) for 20 sec in lysis buffer (1:15; 15 μl of 1×phosphate-buffered saline, pH 7.3, with 2% HALT, 0.15% NP-40, 0.1% ascorbic acid per 1 μg of tissue). Each sample remained on ice for 5 min before spinning at 17,200 g for 20 min at 4° C.; the supernatant was placed in a new tube. Serotonin was detected in individual samples in duplicate with a commercially available serotonin ELISA kit (ADI-900-175, Enzo Life Sciences, Farmingdale, N.Y.) according to the manufacturer directions. Serotonin levels were normalized to the protein concentration for each homogenized sample.

Biotinylation of surface proteins. Ten minutes after fear conditioning, the BLA was microdissected and the tissue was processed for biotinylation of surface proteins using a protocol developed for hippocampal slices [66] and BLA punches [67]. Mice were overdosed with isoflurane and the brain was rapidly dissected and placed into chilled 0.1 M phosphate-buffered saline (PBS; pH 7.4) for one minute. After placement in a chilled matrix, 1 mm thick coronal sections were taken. Bilateral punches (2 mm diameter) containing the BLA were removed from each mouse and coarsely minced. Each tissue mince was placed into 500 μl of ice-cold Tris-Buffered Saline (pH 7.2) containing 5% HALT and placed on ice. Pairs of samples were processed for surface biotinylation using a commercial kit (Pierce Biotechnology, Rockford, Ill.) according to manufacturer instructions. After sample elution, the protein concentration of each sample was determined and the remaining sample was aliquoted and placed at −80° C. for storage.

Protein assay. Protein concentrations of tissue homogenates were determined in duplicate using a commercial kit (Thermo Fisher Scientific, Inc., Waltham, Mass.). Manufacturer's instructions for the microplate assay procedure were followed except that a sufficient volume for two wells of standard (20 μl) or unknown protein solution (20 μl of either a 1:10 or 1:5 dilution in sterile water) was combined with two wells of protein assay reagent (300 μl) in a single Eppendorf tube before 160 μl was pipetted into each well of the microplate. For biotinylated tissue samples, Ionic Detergent Compatibility Reagent (Thermo Fisher Scientific, Inc., Waltham, Mass.) was added to the protein assay reagent (5% w/v) before combining this reagent with the standards and samples. Western blot. Protein samples (8 μg for 5-HT2CR and 30 μg for adenosine deaminase acting on RNA 1 (ADAR1)) were heated to 95° C. for 10 min, and loaded into a standard polyacrylamide gel (NuPAGE Bis-Tris 4-12%; Life Technologies, Grand Island, N.Y.). Protein was transferred to a nitrocellulose membrane electrophoretically using the iBlot dry-blotting system (175 V for 75 min; Life Technologies). Nonspecific binding was reduced with Odyssey blocking buffer for 1 h at room temperature (RT). Primary antibodies (in Odyssey blocking buffer containing 0.2% Tween-20 overnight at 4° C.) were: rabbit anti-5-HT2CR (1:5,000; LifeSpan BioSciences, Seattle, Wash.) and rabbit anti-ADAR1 (1:1,000; Cell Applications, San Diego, Calif.). The loading control for samples was mouse anti-β-actin (1:200,000; Sigma-Aldrich). Blots were washed 4×5 min with PBS with 0.1% Tween-20, and probed with IRDye 800CW goat anti-rabbit and goat anti-mouse IgG secondary antibodies (1:10,000; LI-COR Biosciences, Lincoln, Nebr.) for 1 h at RT. Each band was detected and quantified by the Odyssey Infrared Imaging System (LI-COR Biosciences). For each sample, the protein level was normalized to the loading control β-actin.

Immunohistochemistry. Following experimentation, mice were anesthetized with isofluorane and perfused through the left cardiac ventricle with ice-cold physiological saline followed by 4% paraformaldehyde in 0.1 M phosphate buffer (PB; pH 7.4). Brains were removed and post-fixed overnight, then transferred to 30% sucrose in PB and stored at 4° C. until sectioning. DRN serial sections (30 μm) were obtained in a −20° C. cryostat and placed in 0.01 M PBS until processing.

Sections were washed three times in PBS containing 0.5% Triton X-100 (PBS-T) and then blocked overnight at 4° C. in PBS-T with 2.5% bovine serum albumin. Then, sections were incubated for 48 h at 4° C. with a mixture of primary antibodies: chicken anti-GFP (1:500; Millipore) and mouse anti-tryptophan hydroxylase (TPH; 1:500, Sigma). Sections were then washed with PBS-T and incubated (2 h) at RT with secondary antibodies conjugated to different dyes: goat anti-chicken Alexa Fluor 488 and goat anti-mouse Alexa Fluor 594 (1:500; Invitrogen). After several washes in PBS the sections were mounted onto SuperFrost Plus slides (Fisher Scientific) and coverslipped with VectaShield mounting medium with DAPI (Vector Laboratories, Burlingame, Calif.) and sealed with nail polish for microscopy.

Tissue was examined on a confocal laser scanning microscope (Carl Zeiss, Jena, Germany) and images of DRN sections were taken by acquiring image stacks as provided by the microscope software for validation of virus injection sites. For quantification of labeling efficiency and colocalization of GFP-expressing and TPH-immunoreactive (ir) neurons, brain sections from GFP-transduced SERT-Cre mice were collected spanning the rostral-caudal axis of the DRN from approximately bregma −4.30 to −4.90 mm. The number of TPH-ir neurons coexpressing GFP, the number of GFP-ir neurons coexpres sing TPH, and the total numbers of TPH- and GFP-ir neurons were counted. For each subject, two brain sections at each rostral-caudal level of the DRN were quantified and averaged. GFP immunofluorescence was not observed in the median raphe nucleus, a serotonergic structure ventral to the DRN.

Statistics. All statistical comparisons were computed using StatView for Windows (Version 5.0.1; SAS Institute, Cary, N.C.). Data were analyzed by either Student's t-test or repeated-measures ANOVA followed by post hoc comparisons (Fisher's protected least significant difference). All data is expressed±standard error of the mean. All group data were considered statistically significant if P<0.05. All results are comprised of two or more independent replications for each experiment.

### EXAMPLE 1

### Stress Enables Reinforcement-Elicited Serotonergic Consolidation of Fear Memory

### Repeated Stress Enhances the Consolidation of Fear Memories Established Under Degraded Contingency

Stress exposure can enhance learned fear memories [6,27,28], modeling the way in which a history of stress exposure can predispose humans to disorders of fear or anxiety [1,29]. Here, we exposed mice to either two days of immobilization stress (Stress; 1 h/day) or handling (No Stress) followed by auditory fear conditioning (FIG. 1). Unlike previous studies that examined the relationship between stress and subsequent auditory fear memory [6,27], we used an auditory fear conditioning protocol in which two of four tone and footshock presentations were explicitly unpaired (50% pairing), thereby reducing the tone-footshock contingency. Such a paradigm may be more sensitive to the effects of stress than a conventional protocol where the pairing is 100% and all occurrences of footshock are predicted by tone presentation [30]. Conditional fear to the tone was assessed in a novel environment either 2 h (short-term memory) or 24 h (long-term memory) after fear conditioning (FIG. 1A).

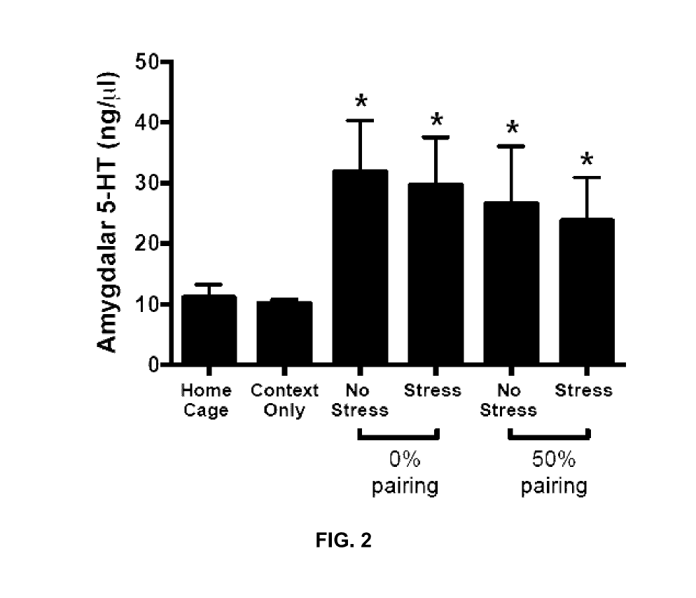

Prior stress did not impact the amount of conditional freezing to the tone during fear acquisition (FIG. 5A) or the short-term memory test (Stress X Tone interaction: F _{(1,19)}=0.384, P=0.543, n=10-11/group, FIG. 1A, left), but did enhance tone-elicited freezing in mice tested 24 h later (Stress X Tone interaction: F _{(1,18)}=11.790, P<0.005; Fisher's PLSD comparing No Stress=37.22±9.22% and Stress=62.78±6.26%, P<0.05, n=10/group, FIG. 1A, right). All groups exhibited comparable and minimal levels of freezing during the 3 min baseline period of the auditory fear test (Fisher's PLSD comparing No Stress to Stress, FIG. 1A, left and right, Ps>0.230), indicating a lack of generalization between the conditioning and testing contexts. Stress did not enhance fear memory via changes in pain processing, general motor activity, or memory retrieval (FIGS. 2, 5B-5D). Enhanced fear memory was also observed only after repeated stress (FIG. 7). The findings that repeated stress enhances long-term but not short-term fear memory when given before fear conditioning suggests that immobilization stress enhances fear responses by strengthening fear memory consolidation.

### Serotonergic Fear Memory Consolidation is Selectively Enabled by Stress

Mice were implanted with bilateral cannulae in the BLA prior to stress or handling. Intra-BLA administration of the highly selective 5-HT2CR antagonist SB242084 (0.4 μg/0.4 μl) [24] immediately following fear conditioning completely blocked stress-induced enhancement of fear when mice were tested for long-term fear memory 24 h later (Stress X Tone interaction: F _{(1,24)}=4.277, P<0.05; Fisher's PLSD comparing Stress-Vehicle=65.08±9.90% and Stress-SB242084=35.56±10.01%, P<0.05, n=6-10/group, FIG. 1B), but did not affect fear levels in the absence of prior stress (Fisher's PLSD comparing No Stress-Vehicle=27.78±5.91% and No Stress-SB242084=19.44±6.21%, P=n.s.). These findings reveal that serotonin-mediated consolidation of fear memory occurs through amygdalar 5-HT2CR and is selectively enabled by a prior history of stress exposure.

### Stress Enhances Amygdala Sensitivity to Serotonin

There are at least two possible mechanisms by which repeated stress may selectively engage serotonergic consolidation of fear memory through 5-HT2CR. One possibility is that stress enhances the release of serotonin from DRN afferents to the BLA during fear conditioning. As an alternative or even concurrent change, it is possible that stress may increase the membrane expression of postsynaptic serotonin receptors in BLA neurons [31,32], leading to enhanced postsynaptic sensitivity to serotonin release by the DRN.

First we determined whether prior stress impacts BLA serotonin levels during conditioning. In addition to the 50% pairing fear conditioning protocol (two tone-shock pairings with two unpaired tones and two unpaired footshocks), a 0% pairing protocol was used (four unpaired tones and four unpaired footshocks). This allowed us to determine whether BLA serotonin levels differ when negative reinforcement is uncoupled from the discrete auditory cue. Two control groups were included: one remained in the home cage until the time of sacrifice (Home Cage group) and the other was placed in the conditioning context, but neither tones nor footshocks were presented (Context Only group). Mice were sacrificed 30 min following fear conditioning, a time point where extracellular serotonin in the amygdala is maximally elevated by the conditioning procedure [14,15].

The serotonin content of the BLA was increased by fear conditioning (Conditioning: F _{(1,36)}32 4.381, P<0.05, n=4-8/group; Fisher's PLSD comparing all Stress and No Stress groups with control groups, P<0.05, FIG. 2), but not exposure to the novel context (Fisher's PLSD comparing Context Only and Home Cage groups, P=n.s., FIG. 2). Within the groups that received fear conditioning, there was no effect of pairing on serotonin levels (Pairing: F _{(1,27)}=0.46, P=n.s., FIG. 2), and, most critically, serotonin was similarly elevated in Stress and No Stress mice (Pairing X Stress: F _{(1,27)}=0.002, P=n.s., n=7-8/group, FIG. 2). Thus, BLA serotonin content is elevated by fear conditioning, but this is not influenced by the prior stress history of the animal. The similar post-conditioning levels of serotonin in subjects receiving the 0% and 50% pairing paradigms also suggests that footshock is the primary factor in determining conditioning-related increases in BLA serotonin.

We next examined the postsynaptic sensitivity of BLA neurons to serotonin following stress by measuring the surface expression of 5-HT2CR in the BLA. Mice received either two days of immobilization stress (Stress groups) or handling (No Stress groups), followed by auditory fear conditioning with 50% pairing. Mice were sacrificed 10 min after fear conditioning ended. 5-HT2CR density was assessed at this post-conditioning time point because it corresponds roughly to both the time when serotonin is first elevated by fear conditioning [14,15] and a time when cellular consolidation of fear memory is occurring [34].

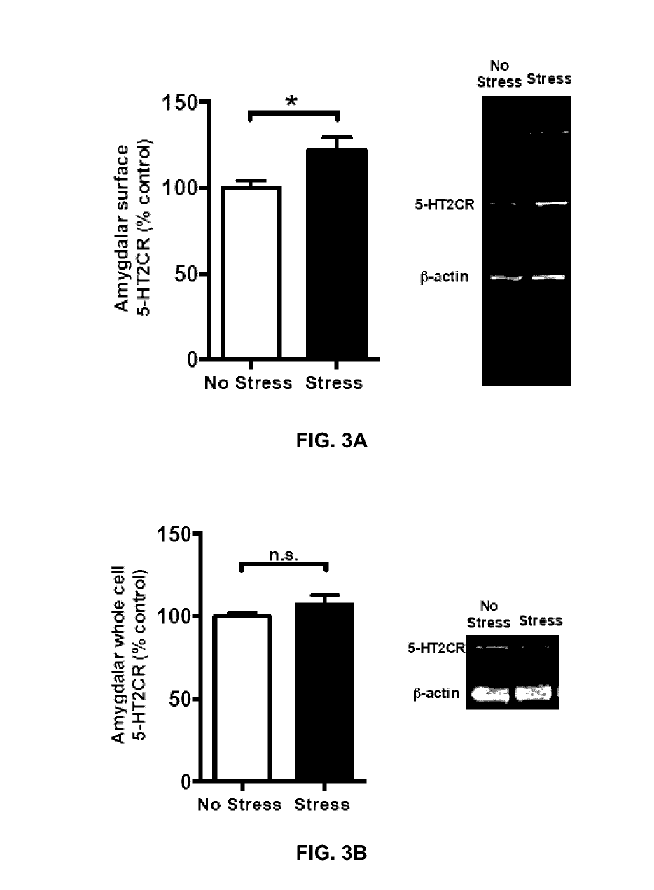

We found that repeated stress produced a significant increase in surface membrane expression of the 5-HT2CR in the amygdala (Stress: F _{(1,26)}=4.887, P<0.05, n=12-16/group, FIG. 3A), without affecting the total pool of 5-HT2CR (Stress: F _{(1,10)}=1.504, P=n.s., n=6/group, FIG. 3B). This finding suggests that repeated stress alters trafficking of 5-HT2CR, as opposed to an upregulation of gene transcription or protein translation. Stress is known to trigger editing of the pre-messenger mRNA for the 5-HT2CR [35] through adenosine deaminase acting on RNA 1 (ADAR1) [36]. Because edited forms of the 5-HT2CR are known to have less internalization from the membrane surface [37], we next examined expression of ADAR1. We found that repeated stress significantly enhances total levels of this protein in the BLA (Stress: F _{(1,10)}=4.975, P<0.05, n=6/group, FIG. 3C). Together, these data show that the amygdala exhibits an enhanced membrane presence of 5-HT2CR following repeated stress.

Stress-Enhanced Fear Cannot be Attributed to Enhanced Acquisition, Pain Processing, or Retrieval

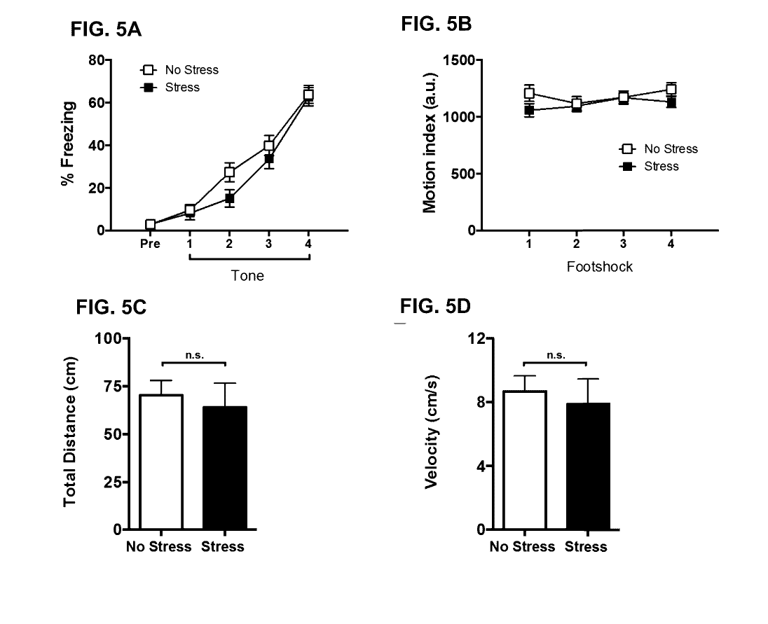

We explored the possibility that repeated stress enhances fear memory by facilitating fear acquisition, potentiating pain processing during conditioning, or enhancing retrieval and/or performance during the long-term memory test. In groups of stressed and unstressed mice, stress had no impact on freezing levels during fear acquisition (Stress X Time interaction: F _{(4,216)}=1.40, P=n.s., FIG. 5A). Thus, repeated stress did not enhance fear memory acquisition. The memory enhancing effect of stress cannot be attributed to stress-related enhancement of pain processing during the aversive footshocks: repeated stress did not alter the motor response to the footshock (Stress X Trial interaction: F _{(3,162)}=0.993, P=n.s., n=27-29/group, FIG. 5B). Furthermore, prior immobilization stress did not alter general motor activity (total distance and velocity) during the pre-tone period prior to the auditory fear test (Ps=n.s., unpaired t-test, FIGS. 5B-5C). It also cannot be attributed to effects of stress on long-term fear memory retrieval or performance because exposure to repeated immobilization stress after fear conditioning had no effect on later fear retrieval (Stress X Tone interaction: F _{(1,18)}=3.415, P=n.s., n=10/group, FIG. 6. The observation that repeated stress initiated 24 h following fear conditioning has no impact on long-term fear memory suggests that an important window in which stress influences consolidation occurs shortly after the fear conditioning.

We also examined whether the immobilization stress had to be repeated to produce enhancement of learned auditory fear. We found that single session of immobilization stress did not produce fear enhancement (Stress X Tone: F _{(1,17)}=0.566, P=n.s., n=9-10/group, FIG. 7).

### REFERENCES

1. Mazure C (1995): Does Stress Cause Psychiatric Illness? Washington, D.C.: American Psychiatric Press, Inc.
2. Schwartz A C, Bradley R L, Sexton M, Sherry A, Ressler K J (2005): Posttraumatic stress disorder among African Americans in an inner city mental health clinic. Psychiatr Sery 56:212-215.
3. Diamond D M, Campbell A M, Park C R, Halonen J, Zoladz P R (2007): The temporal dynamics model of emotional memory processing: a synthesis on the neurobiological basis of stress-induced amnesia, flashbulb and traumatic memories, and the Yerkes-Dodson law. Neural Plast 2007:60803.
4. Glover E M, Phifer J E, Crain D F, Norrholm S D, Davis M, Bradley B, et al. (2011): Tools for translational neuroscience: PTSD is associated with heightened fear responses using acoustic startle but not skin conductance measures. Depress Anxiety 28:1058-1066.
5. Roy M J, Costanzo M E, Jovanovic T, Leaman S, Taylor P, Norrholm S D, et al. (2013): Heart rate response to fear conditioning and virtual reality in subthreshold PTSD. Stud Health Technol Inform 191:115-119.
6. Meyer R M, Burgos-Robles A, Liu E, Correia S S, Goosens K A (2013): A ghrelin-growth hormone axis drives stress-induced vulnerability to enhanced fear. Mol Psychiatry.
7. Suvrathan A, Bennur S, Ghosh S, Tomar A, Anilkumar S, Chattarji S (2014): Stress enhances fear by forming new synapses with greater capacity for long-term potentiation in the amygdala. Philos Trans R Soc Lond B Biol Sci 369:20130151.
8. Yehuda R (2002): Post-traumatic stress disorder. N Engl J Med 346:108-114.
9. Lowry C A, Hale M W, Evans A K, Heerkens J, Staub D R, Gasser P J, et al. (2008): Serotonergic systems, anxiety, and affective disorder: focus on the dorsomedial part of the dorsal raphe nucleus. Ann N Y Acad Sci 1148:86-94.
10. Armbruster D, Moser D A, Strobel A, Hensch T, Kirschbaum C, Lesch K P, et al. (2009): Serotonin transporter gene variation and stressful life events impact processing of fear and anxiety. Int J Neuropsychopharmacol 12:393-401.
11. Ranade S P, Mainen Z F (2009): Transient firing of dorsal raphe neurons encodes diverse and specific sensory, motor, and reward events. J Neurophysiol 102:3026-3037.
12. Schweimer J V, Ungless M A (2010): Phasic responses in dorsal raphe serotonin neurons to noxious stimuli. Neuroscience 171:1209-1215.
13. Waterhouse B D, Devilbiss D, Seiple S, Markowitz R (2004): Sensorimotor-related discharge of simultaneously recorded, single neurons in the dorsal raphe nucleus of the awake, unrestrained rat. Brain Res 1000:183-191.
14. Yokoyama M, Suzuki E, Sato T, Maruta S, Watanabe S, Miyaoka H (2005): Amygdalic levels of dopamine and serotonin rise upon exposure to conditioned fear stress without elevation of glutamate. Neurosci Lett 379:37-41.
15. Zanoveli J M, Carvalho M C, Cunha J M, Brandao M L (2009): Extracellular serotonin level in the basolateral nucleus of the amygdala and dorsal periaqueductal gray under unconditioned and conditioned fear states: an in vivo microdialysis study. Brain Res 1294:106-115.
16. Bonn M, Schmitt A, Lesch K P, Van Bockstaele E J, Asan E (2013): Serotonergic innervation and serotonin receptor expression of NPY-producing neurons in the rat lateral and basolateral amygdaloid nuclei. Brain Struct Funct 218:421-435.
17. Van Veen J F, Van der Wee N J, Fiselier J, Van Vliet I M, Westenberg H G (2007): Behavioural effects of rapid intravenous administration of meta-chlorophenylpiperazine (m-CPP) in patients with generalized social anxiety disorder, panic disorder and healthy controls. Eur Neuropsychopharmacol 17:637-642.
18. Li Q, Luo T, Jiang X, Wang J (2012): Anxiolytic effects of 5-HT(1)A receptors and anxiogenic effects of 5-HT(2)C receptors in the amygdala of mice. Neuropharmacology 62:474-484.
19. Christianson J P, Ragole T, Amat J, Greenwood B N, Strong P V, Paul E D, et al. (2010): 5-hydroxytryptamine 2C receptors in the basolateral amygdala are involved in the expression of anxiety after uncontrollable traumatic stress. Biol Psychiatry 67:339-345.
20. Michelsen K A, Schmitz C, Steinbusch H W (2007): The dorsal raphe nucleus—from silver stainings to a role in depression. Brain Res Rev 55:329-342.
21. Vertes R P (1991): A PHA-L analysis of ascending projections of the dorsal raphe nucleus in the rat. J Comp Neurol 313:643-668.
22. Hale M W, Shekhar A, Lowry C A (2012): Stress-related serotonergic systems: implications for symptomatology of anxiety and affective disorders. Cell Mol Neurobiol 32:695-708.
23. Cools R, Roberts A C, Robbins T W (2008): Serotoninergic regulation of emotional and behavioural control processes. Trends Cogn Sci 12:31-40.
24. Kennett G A, Wood M D, Bright F, Trail B, Riley G, Holland V, et al. (1997): SB 242084, a selective and brain penetrant 5-HT2C receptor antagonist. Neuropharmacology 36:609-620.
25. Zhuang X, Masson J, Gingrich J A, Rayport S, Hen R (2005): Targeted gene expression in dopamine and serotonin neurons of the mouse brain. J Neurosci Methods 143:27-32.
26. Margrie T W, Brecht M, Sakmann B (2002): In vivo, low-resistance, whole-cell recordings from neurons in the anaesthetized and awake mammalian brain. Pflugers Arch 444:491-498.
27. Rau V, DeCola J P, Fanselow M S (2005): Stress-induced enhancement of fear learning: an animal model of posttraumatic stress disorder. Neurosci Biobehav Rev 29:1207-1223.
28. Rau V, Fanselow M S (2009): Exposure to a stressor produces a long lasting enhancement of fear learning in rats. Stress 12:125-133.
29. Lederbogen F, Kirsch P, Haddad L, Streit F, Tost H, Schuch P, et al. (2011): City living and urban upbringing affect neural social stress processing in humans. Nature 474:498-501.
30. Tsetsenis T, Ma X H, Lo Iacono L, Beck S G, Gross C (2007): Suppression of conditioning to ambiguous cues by pharmacogenetic inhibition of the dentate gyrus. Nat Neurosci 10:896-902.
31. Magalhaes A C, Holmes K D, Dale L B, Comps-Agrar L, Lee D, Yadav P N, et al. CRF receptor 1 regulates anxiety behavior via sensitization of 5-HT2 receptor signaling. Nat Neurosci 13:622-629.
32. Flugge G (2000): Regulation of monoamine receptors in the brain: dynamic changes during stress. Int Rev Cytol 195:145-213.
33. Bouchez G, Millan M J, Rivet J M, Billiras R, Boulanger R, Gobert A (2012): Quantification of extracellular levels of corticosterone in the basolateral amygdaloid complex of freely-moving rats: a dialysis study of circadian variation and stress-induced modulation. Brain Res 1452:47-60.
34. Abel T, Lattal K M (2001): Molecular mechanisms of memory acquisition, consolidation and retrieval. Curr Opin Neurobiol 11:180-187.
35. Englander M T, Dulawa S C, Bhansali P, Schmauss C (2005): How stress and fluoxetine modulate serotonin 2C receptor pre-mRNA editing. The Journal of neuroscience: the official journal of the Society for Neuroscience 25:648-651.
36. Liu Y, Emeson R B, Samuel C E (1999): Serotonin-2C receptor pre-mRNA editing in rat brain and in vitro by splice site variants of the interferon-inducible double-stranded RNA-specific adenosine deaminase ADAR1. J Biol Chem 274:18351-18358.
37. Marion S, Weiner D M, Caron M G (2004): RNA editing induces variation in desensitization and trafficking of 5-hydroxytryptamine 2c receptor isoforms. J Biol Chem 279:2945-2954.
38. Moya P R, Fox M A, Jensen C L, Laporte J L, French H T, Wendland J R, et al. (2011): Altered 5-HT2C receptor agonist-induced responses and 5-HT2C receptor RNA editing in the amygdala of serotonin transporter knockout mice. BMC Pharmacol 11:3.
39. Wilkinson L O, Jacobs B L (1988): Lack of response of serotonergic neurons in the dorsal raphe nucleus of freely moving cats to stressful stimuli. Exp Neurol 101:445-457.
40. Chow B Y, Han X, Dobry A S, Qian X, Chuong A S, Li M, et al. (2010): High-performance genetically targetable optical neural silencing by light-driven proton pumps. Nature 463:98-102.
41. Davis M, Cassella J V, Kehne J H (1988): Serotonin does not mediate anxiolytic effects of buspirone in the fear-potentiated startle paradigm: comparison with 8-OH-DPAT and ipsapirone. Psychopharmacology (Berl) 94:14-20.
42. Maier S F, Grahn R E, Kalman B A, Sutton L C, Wiertelak E P, Watkins L R (1993): The role of the amygdala and dorsal raphe nucleus in mediating the behavioral consequences of inescapable shock. Behav Neurosci 107:377-388.
43. Rescorla R A (1968): Probability of shock in the presence and absence of CS in fear conditioning. Journal of comparative and physiological psychology 66:1-5.
44. Rescorla R A (1966): Predictability and number pairings in Pavlovian fear conditioning. Psychonomic Science 4:383-384.
45. Cain C K, Godsil B P, Jami S, Barad M (2005): The L-type calcium channel blocker nifedipine impairs extinction, but not reduced contingency effects, in mice. Learn Mem 12:277-284.
46. Witnauer J E, Miller R R (2007): Degraded contingency revisited: posttraining extinction of a cover stimulus attenuates a target cue's behavioral control. J Exp Psychol Anim Behav Process 33:440-450.
47. Mackintosh N J (1975): A theory of attention: Variations in the associability of stimuli with reinforcements. Psychol Rev 82:276-298.
48. Rescorla R A, Wagner A R (1972): A theory of Pavlovian conditioning: Variations in the effectiveness of reinforcement and nonreinforcement. In: Prokasy AHBWF, editor. Classical Conditioning II: Current Research and Theory. New York: Appleton-Century-Crofts, pp 64-99.
49. Miller R R, Barnet R C, Grahame N J (1995): Assessment of the Rescorla-Wagner model. Psychol Bull 117:363-386.
50. Li S S, McNally G P (2014): The conditions that promote fear learning: prediction error and Pavlovian fear conditioning. Neurobiol Learn Mem 108:14-21.
51. Hermann A, Kupper Y, Schmitz A, Walter B, Vaitl D, Hennig J, et al. (2012): Functional gene polymorphisms in the serotonin system and traumatic life events modulate the neural basis of fear acquisition and extinction. PLoS One 7:e44352.
52. Mekli K, Payton A, Miyajima F, Platt H, Thomas E, Downey D, et al. (2011): The HTR1A and HTR1B receptor genes influence stress-related information processing. Eur Neuropsychopharmacol 21:129-139.
53. American Psychiatric Association (2013): Diagnostic and statistical manual of mental disorders (5th ed.). Arlington, Va.: American Psychiatric Publishing.
54. Hackmann A, Ehlers A, Speckens A, Clark D M (2004): Characteristics and content of intrusive memories in PTSD and their changes with treatment. J Trauma Stress 17:231-240.
55. Catani C, Jacob N, Schauer E, Kohila M, Neuner F (2008): Family violence, war, and natural disasters: a study of the effect of extreme stress on children's mental health in Sri Lanka. BMC Psychiatry 8:33.
56. De Berardis D, Serroni N, Marini S, Moschetta F S, Martinotti G, Di Giannantonio M (2012): Agomelatine for the treatment of posttraumatic stress disorder: a case report. Ann Clin Psychiatry 24:241-242.
57. Boyden E S, Raymond J L (2003): Active reversal of motor memories reveals rules governing memory encoding. Neuron 39:1031-1042.
58. Han X, Chow B Y, Zhou H, Klapoetke N C, Chuong A, Rajimehr R, et al. (2011): A high-light sensitivity optical neural silencer: development and application to optogenetic control of non-human primate cortex. Front Syst Neurosci 5.
59. Aghajanian G K, Vandermaelen C P (1982): Intracellular recordings from serotonergic dorsal raphe neurons: pacemaker potentials and the effect of LSD. Brain Res 238:463-469.
60. Allers K A, Sharp T (2003): Neurochemical and anatomical identification of fast-and slow-firing neurones in the rat dorsal raphe nucleus using juxtacellular labelling methods in vivo. Neuroscience 122:193-204.
61. Hajos M, Allers K A, Jennings K, Sharp T, Charette G, Sik A, et al. (2007): Neurochemical identification of stereotypic burst-firing neurons in the rat dorsal raphe nucleus using juxtacellular labelling methods. Eur J Neurosci 25:119-126.
62. Schweimer J V, Mallet N, Sharp T, Ungless M A (2011): Spike-timing relationship of neurochemically-identified dorsal raphe neurons during cortical slow oscillations. Neuroscience 196:115-123.
63. Chegini H R, Nasehi M, Zarrindast M R (2014): Differential role of the basolateral amygdala 5-HT3 and 5-HT4 serotonin receptors upon ACPA-induced anxiolytic-like behaviors and emotional memory deficit in mice. Behavioural brain research 261:114-126.
64. Takahashi A, Shimamoto A, Boyson C O, DeBold J F, Miczek K A (2010): GABA(B) receptor modulation of serotonin neurons in the dorsal raphe nucleus and escalation of aggression in mice. The Journal of neuroscience: the official journal of the Society for Neuroscience 30:11771-11780.
65. Blanchard R J, Blanchard D C (1969): Crouching as an index of fear. Journal of comparative and physiological psychology 67:370-375.
66. Chung H, Xia J, Scannevin R, Zhang X, Huganir R (2000): Phosphorylation of the AMPA receptor subunit GluR2 differentially regulates its interaction with PDZ domain-containing proteins. J Neurosci 21:7258-7267.
67. Kim J, Lee S, Park K, Hong I, Song B, Son G, et al. (2007): Amygdala depotentiation and fear extinction. Proc Natl Acad Sci U S A 104:20955-20960.
68. Rodriguez Manzanares P A, Isoardi N A, Caner H F, Molina V A (2005): Previous stress facilitates fear memory, attenuates GABAergic inhibition, and increases synaptic plasticity in the rat basolateral amygdala. J Neurosci 25:8725-8734.
69. Roozendaal B, McGaugh J L (2011): Memory modulation. Behav Neurosci 125:797-824.
70. Gold P E (2014): Regulation of memory—From the adrenal medulla to liver to astrocytes to neurons. Brain Res Bull.
71. Hale M W, Hay-Schmidt A, Mikkelsen J D, Poulsen B, Bouwknecht J A, Evans A K, et al. (2008): Exposure to an open-field arena increases c-Fos expression in a subpopulation of neurons in the dorsal raphe nucleus, including neurons projecting to the basolateral amygdaloid complex. Neuroscience 157:733-748.
72. Coplan J D, Andrews M W, Rosenblum L A, Owens M J, Friedman S, Gorman J M, et al. (1996): Persistent elevations of cerebrospinal fluid concentrations of corticotropin-releasing factor in adult nonhuman primates exposed to early-life stressors: implications for the pathophysiology of mood and anxiety disorders. Proc Natl Acad Sci U S A 93:1619-1623.
73. Hansson C, Alvarez-Crespo M, Taube M, Skibicka K P, Schmidt L, Karlsson-Lindahl L, et al. (2014): Influence of ghrelin on the central serotonergic signaling system in mice. Neuropharmacology 79:498-505.
74. Schellekens H, Dinan T G, Cryan J F (2013): Taking two to tango: a role for ghrelin receptor heterodimerization in stress and reward. Front Neurosci 7:148.
75. Schellekens H, van Oeffelen W E, Dinan T G, Cryan J F (2013): Promiscuous dimerization of the growth hormone secretagogue receptor (GHS-R1a) attenuates ghrelin-mediated signaling. J Biol Chem 288:181-191.
76. Wang Q, O'Brien P J, Chen C X, Cho D S, Murray J M, Nishikura K (2000): Altered G protein-coupling functions of RNA editing isoform and splicing variant serotonin2C receptors. J Neurochem 74:1290-1300.
77. Niswender C M, Copeland S C, Herrick-Davis K, Emeson R B, Sanders-Bush E (1999): RNA editing of the human serotonin 5-hydroxytryptamine 2C receptor silences constitutive activity. J Biol Chem 274:9472-9478.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

1. A method for preventing a stress-associated disorder comprising administering to a subject at risk of having a stress-associated disorder a serotonin 2c receptor (5-HT2CR) antagonist prior to, during, or within an hour following a stress-related event in an effective amount to prevent the stress-associated disorder.

2. The method of claim 1, wherein the stress-associated disorder is post-traumatic stress disorder (PTSD).

3. The method of claim 1, wherein the 5-HT2CR antagonist is agomelatine.

4. The method of claim 1, wherein the 5-HT2CR antagonist is SB 242084, RS 102221 hydrochloride, SB 206553 {5-methyl-1-[(3-pyridylcarba-moyl)-1,2,3,5-tetrahydropyrrolo(2,3-f)indole]}, SB 206553 hydrochloride, SB 200646A [N-(1-methyl-5-indolyl)-N′-(3-pyridyl) urea hydrochloride], SB 200646 hydrochloride, clozapine, N-Desmethylclozapine, mesulergine hydrochloride, S 32212 hydrochloride, SB 221284, SDZ SER 082 fumarate, or analogs thereof.

5. The method of claim 1, wherein the 5-HT2CR antagonist is administered to the subject in an amount of 25 to 50 mg per day.

6. The method of claim 1, wherein the 5-HT2CR antagonist is administered orally.

7. The method of claim 1, wherein the 5-HT2CR antagonist is administered daily beginning one week prior to the stress-related event.

8. The method of claim 1, wherein the 5-HT2CR antagonist is administered throughout the duration of the stress-related event.

9. The method of claim 1, wherein the 5-HT2CR antagonist is administered for up to 24 weeks after the stress-related event.

10. A method for treating PTSD comprising administering to a subject having PTSD a 5-HT2CR antagonist in conjunction with a cognitive therapy for memory reconsolidation.

11. The method of claim 10, wherein the 5-HT2CR antagonist is administered during the cognitive therapy for memory reconsolidation.

12. The method of claim 10, wherein the 5-HT2CR antagonist is administered within 24 hours of the cognitive therapy for memory reconsolidation.

13. The method of claim 10, wherein the 5-HT2CR antagonist is administered within 1 week of the cognitive therapy for memory reconsolidation.

14-18. (canceled)

19. The method of claim 10, wherein the 5-HT2CR antagonist is an inhibitory nucleic acid.

20. The method of claim 10, wherein the 5-HT2CR antagonist is a 5HT2c receptor inverse agonist.

21. The method of claim 20, wherein the 5-HT2CR inverse agonist is SB 228357 (N-[3-Fluoro-5-(3-pyrindyl)phenyl]-2,3-dihydro-5-methoxy-6-(trifluoromethyl)-1H-indole-1-carboxamide) or SB 243213 dihydrochloride (2,3-Dihydro-5-methyl-N-[6-[(2-methyl-3-pyridinyl)oxy]-3-pyridinyl]-6-(trifluoromethyl)-1H-Indole-1-carboxamide dihydrochloride).

22. The method of claim 10, wherein the 5-HT2CR antagonist is a clozapine metabolite.

23. The method of claim 22, wherein the clozapine metabolite is clozapine N-oxide.

24. A method for treating or preventing a stress-associated disorder comprising administering to a subject having a stress-associated disorder a 5-HT2CR antagonist in an effective amount to treat the stress-associated disorder, wherein the 5-HT2CR antagonist is not agomelatine.

25-30. (canceled)

31. The method of claim 1, further comprising identifying a subject who has enhanced fear associated with multiple stresses or traumatic memory strength.

32-43. (canceled)
